(19) United States
(12) Patent Application Publication
(10) Pub. No.: US 2021/0158588 A1
(43) Pub. Date: May 27, 2021

(54) DATA-CREATION ASSISTANCE APPARATUS, DATA-CREATION ASSISTANCE METHOD, AND DATA-CREATION ASSISTANCE PROGRAM

(71) Applicant: OLYMPUS CORPORATION, Tokyo (JP)

(72) Inventors: Takashi MIYOSHI, Tokyo (JP); Isao SAKANE, Tokyo (JP); Taiji MINE, Tokyo (JP); Takayuki NAKATOMI, Tokyo (JP)

(73) Assignee: OLYMPUS CORPORATION, Tokyo (JP)

(21) Appl. No.: 17/164,123

(22) Filed: Feb. 1, 2021

Related U.S. Application Data
(63) Continuation of application No. PCT/JP2018/029322, filed on Aug. 6, 2018.

(51) Int. Cl. G06T 11/60; G06K 9/00; G06T 7/11; G06T 7/62

(52) U.S. Cl. CPC G06T 11/60; G06K 9/00147; G06T 7/11; G06T 7/62; G06T 2210/41; G06K 2209/27; G06K 2209/05; G06T 2207/20092; G06T 2207/30024

(57) ABSTRACT

Provided is a data-creation assistance apparatus which is an apparatus that assists data creation work in which an annotation is added to each cell in an image containing cells, the data-creation assistance apparatus including: a processor including hardware, the processor being configured to: acquire image data including the image; extract information related to the cells from the acquired image data; set a size of divided regions on the basis of the extracted information related to the cells; and divide the image into the divided regions; and a display that displays each of the divided regions which have been divided.

### CROSS-REFERENCE TO RELATED APPLICATIONS

This is a continuation of International Application PCT/JP2018/029322 which is hereby incorporated by reference herein in its entirety.

### TECHNICAL FIELD

The present invention relates to a data-creation assistance apparatus, a data-creation assistance method, and a data-creation assistance program.

### BACKGROUND ART

There is a known image processing apparatus that displays annotations that an operator has added to a virtual slide image (for example, see Patent Literature 1).

Annotation task in which correct-solution data indicating the position and the state of a target object in an image are manually created or corrected is performed while enlarging or shrinking the image depending on the size and features of the target object.

### CITATION LIST

### Patent Literature

{Patent Literature 1} Publication of Japanese Patent No. 6091137

### SUMMARY OF INVENTION

An aspect of the present invention is a data-creation assistance apparatus which is an apparatus that assists data creation work in which an annotation is added to each cell in an image containing cells, the data-creation assistance apparatus including: a processor including hardware, the processor being configured to: acquire image data including the image; extract information related to the cells from the acquired image data; set a size of divided regions on the basis of the extracted information related to the cells; and divide the image into the divided regions; and a display that displays each of the divided regions which have been divided.

Another aspect of the present invention is a data-creation assistance method that assists data creation work in which an annotation is added to each cell in an image containing cells, the data-creation assistance method including: acquiring image data including the image; extracting information related to the cells from the acquired image data; setting a size of divided regions on the basis of the extracted information related to the cells; dividing the image into the divided regions; and displaying each of the divided regions which have been divided.

Another aspect of the present invention is a non-transitory computer-readable medium having a data-creation assistance program stored therein, the data-creation assistance program assisting data creation work in which an annotation is added to each cell in an image containing cells and causing a computer to execute function of: acquiring image data including the image; extracting information related to the cells from the acquired image data; setting a size of divided regions on the basis of the extracted information related to the cells; dividing the image into the divided regions; and displaying each of the divided regions which have been divided.

### BRIEF DESCRIPTION OF DRAWINGS

FIG. 1 FIG. 1 is a block diagram showing a data-creation assistance apparatus according to an embodiment of the present invention.

FIG. 2 FIG. 2 is a diagram showing example divided regions of an image created by the data-creation assistance apparatus in FIG. 1.

FIG. 3 FIG. 3 is an image example showing an example annotation.

FIG. 4 FIG. 4 is an image example showing an example annotation.

FIG. 5 FIG. 5 is an image example showing an example annotation.

FIG. 6 FIG. 6 is an image example showing an example annotation.

FIG. 7 FIG. 7 is an image example showing an example annotation.

FIG. 8 FIG. 8 is an image example showing an example annotation.

FIG. 9 FIG. 9 is a flowchart showing a data-creation assistance method employing the data-creation assistance apparatus in FIG. 1.

FIG. 10 FIG. 10 is a block diagram showing a modification of the data-creation assistance apparatus in FIG. 1.

FIG. 11 FIG. 11 is a diagram showing the display sequence of the divided regions created by another modification of the data-creation assistance apparatus in FIG. 1.

FIG. 12 FIG. 12 is a diagram showing a display example of the divided regions created by another modification of the data-creation assistance apparatus in FIG. 1.

### DESCRIPTION OF EMBODIMENT

A data-creation assistance apparatus 1 and a data-creation assistance method according to an embodiment of the present invention will be described below with reference to the drawings.

The data-creation assistance apparatus 1 according to this embodiment is an apparatus that assists data creation work (hereinafter also referred to as annotation work) in which, in an image G containing cells X, an annotation is added to each of the cells X.

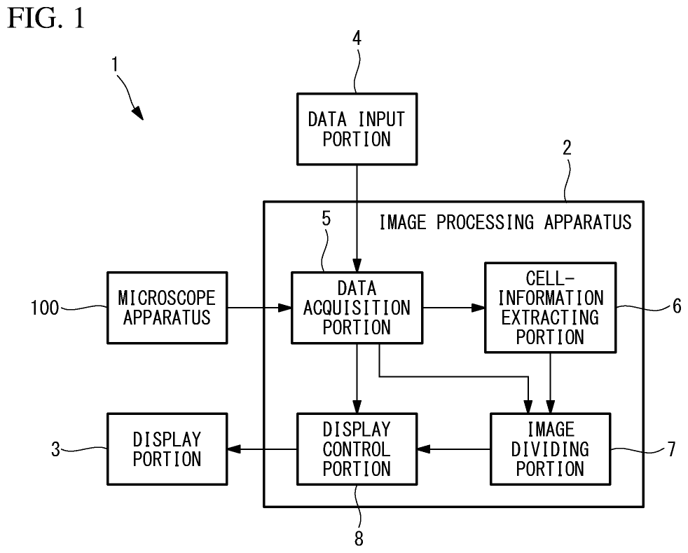

As shown in FIG. 1, the data-creation assistance apparatus 1 according to this embodiment includes: an image processing apparatus 2 that processes the image G acquired by a microscope apparatus 100; a display portion 3 that displays the processed image G; and a data input portion (input portion) 4 with which an operator inputs annotations or the like.

The image processing apparatus 2 includes: a data acquisition portion (image-data acquisition portion) 5 that acquires image data containing the image G acquired by the microscope apparatus 100; a cell-information extracting portion 6 that extracts information related to the density of the cells X from the acquired image data; an image dividing portion 7 that determines the size of a divided region A on the basis of the extracted information related to the density of the cells X and that divides the image G into one or more divided regions A; and a display control portion 8 that causes the display portion 3 to display one of the divided regions A that have been divided. The information related to the density of the cells X includes at least one of the size of the cells X, the density of the cells X, and the type of the cells X. In addition, as the information related to the density of the cells X, it is preferable that at least one of the size of the cells X and the type of the cells X be included in addition to the density of the cells X. In addition, as the density of the cells X, it may be determined that the cells X are present in portions in which the background that does not contain the cells X is inverted and, on the basis of this information, the information related to the density of the cells X may be obtained.

The data acquisition portion 5 acquires metadata from the microscope apparatus 100 together with the image G acquired by the microscope apparatus 100. The metadata include information items such as the type of the cells X and the magnification of the microscope apparatus 100.

The cell-information extracting portion 6 includes standard cell size data in the form of a table, reads out, from the table, a cell size that is closest to the cell size calculated on the basis of the type of the cells X and the magnification of the microscope apparatus 100 included in the metadata, and estimates the size of the cells in the image. Accordingly, the number of pixels in the image G by which the cell size is represented is computed.

The image dividing portion 7 sets, on the basis of the cell size calculated by the cell-information extracting portion 6, the size of the divided region A that encompasses a range from a width corresponding to the size of a single cell X, at a minimum, to a width corresponding to several tens of the cells X and divides the image G into the divided regions A of the set size. The size of each divided region A is, for example, a size at which, when a single divided region A is displayed on the display portion 3 in an enlarged state, it is possible to ascertain the state of the cells X without enlarging or shrinking the display and it is possible to add annotations thereto.

For example, with cells X in which differences between long diameters and short diameters thereof are small, a preferable size is such that 10 to 50 cells X are arrayed in a single divided region A. For example, with cells X in which differences between long diameters and short diameters thereof are large, a preferable size is such that 10 to 50 cells X are arrayed in a single divided region A in terms of the short diameters and that 5 to 25 cells X are arrayed in a single divided region A in terms of the long diameters.

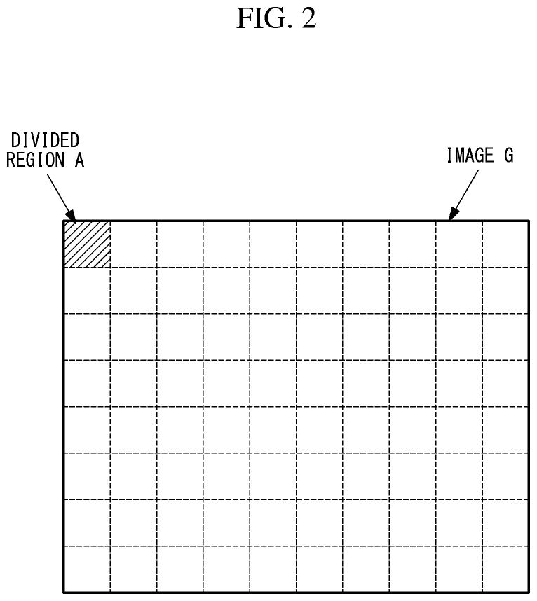

As shown in FIG. 2, the number into which the image G is divided is set by equally dividing the image G vertically and laterally on the basis of a divided region A encompassing the above-described sizes.

In the case in which the density of the cells X is high, the number of annotation work tasks in a single divided region A may be decreased by setting the size of the divided region A so as to take a value for which the number of the cells X is on a lower limit side in the above-described range.

The display control portion 8 enlarges a single divided region A that has been divided by the image dividing portion 7 and displays the divided region A on the display portion 3.

With the data input portion 4, the operator inputs annotations to the image G of the displayed divided region A. The display control portion 8 displays the input annotations on the divided region A in a superimposed manner.

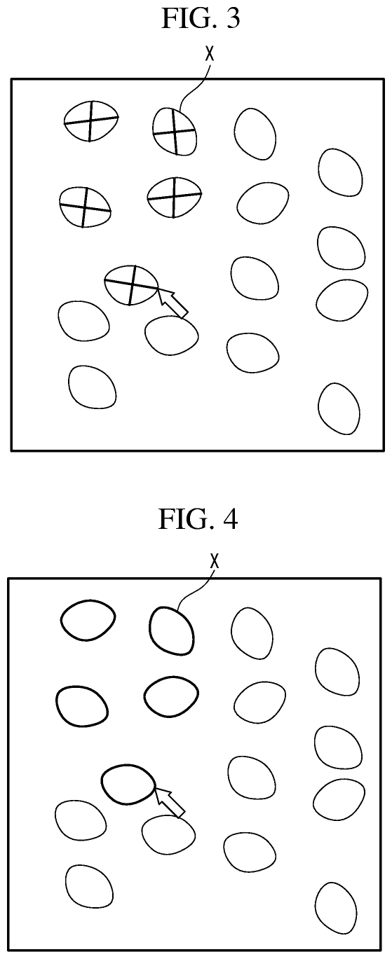

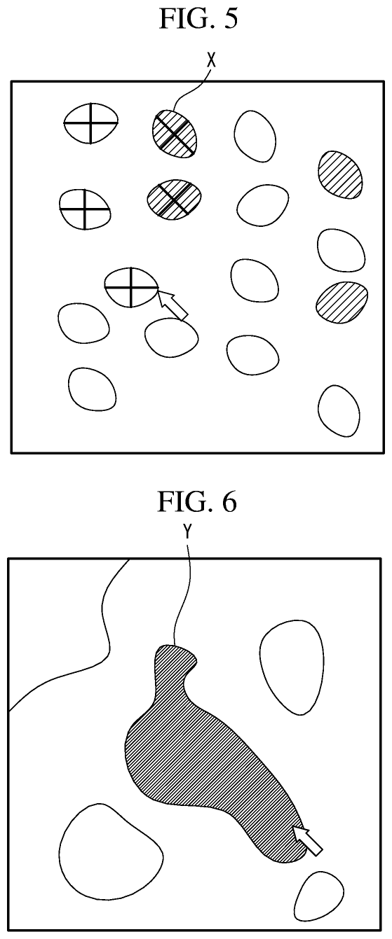

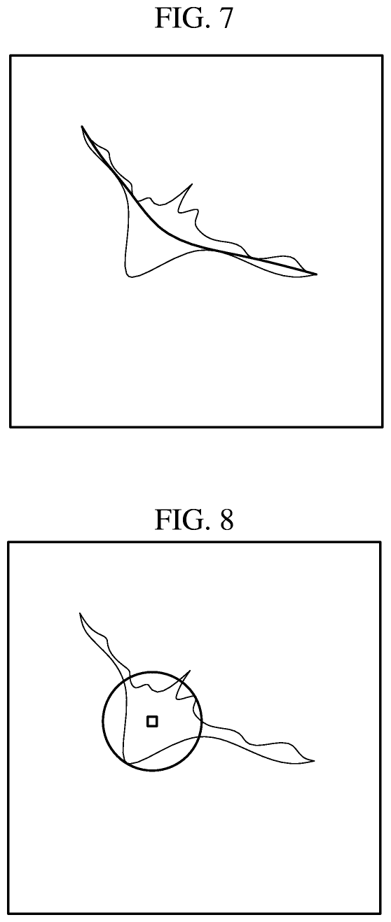

Examples of the annotations include: cross-shaped position markers that the operator adds to points that he/she considers to be centers of the cells X, as shown in FIG. 3; closed-line outline markers that the operator adds to outlines that he/she considers to be boundaries between the cells X and the background, as shown in FIG. 4; cross-shaped or X-shaped position markers that the operator adds to points that he/she considers to be centers of the cells X in accordance with the cell type, as shown in FIG. 5; a region marker in which the operator fills in an area that he/she considers to be a region of a colony Y, as shown in FIG. 6; a pathway maker where the operator adds an open line in a shape in which he/she considers that a pseudopodium of a cell X extends, as shown in FIG. 7; and a marker that the operator adds to indicate the center of a cell X and to indicate, by means of a circle, the size of a principal portion of the cell X, as shown in FIG. 8.

A data-creation assistance method employing the thus-configured data-creation assistance apparatus 1 according to this embodiment will be described below.

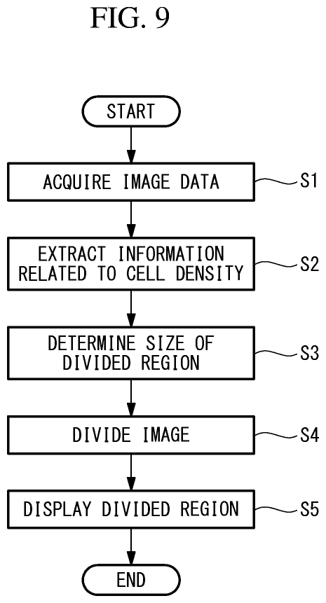

As shown in FIG. 9, with the data-creation assistance method according to this embodiment, the image G is acquired by the microscope apparatus 100, and the metadata are input to the data acquisition portion 5 of the image processing apparatus 2 together with the acquired image G.

Then, the data input portion 4 acquires the image data including the input image G and metadata (step S1), and the cell-information extracting portion 6 estimates the cell size with the image size by reading out the closest cell size to the cell size calculated on the basis of the acquired image data and extracts the information related to the density of the cells X (step S2).

Next, on the basis of the extracted information related to the density of the cells X, for example, the cell size calculated by the cell-information extracting portion 6, the image dividing portion 7 sets the size of the divided region A (step S3) and divides the image G into the divided regions A having the set size (step S4).

Then, the display control portion 8 performs control so as to display, on the display portion 3, a single divided region A, which has been divided, in an enlarged state. Accordingly, for each of the divided regions A, the single divided region A is displayed on the display portion 3 (step S5).

With the data-creation assistance apparatus 1 and the data-creation assistance method according to this embodiment, the image G is divided into one or more divided regions A on the basis of the cell size and each of the divided regions A is set to a size at which it is possible to ascertain the state of the cells X without enlarging or shrinking the display and it is possible to add annotations thereto; therefore, it is not necessary to enlarge or shrink the image G while performing the annotation work, and there is an advantage in that it is possible to perform the annotation work at a constant quality by reducing omissions in the annotation work with respect to the image G containing the cells X.

In this embodiment, the size of the divided region A is set on the basis of the metadata, which are transmitted from the microscope apparatus 100 together with the image G; however, alternatively, cell regions may be identified by applying image processing to the image G transmitted from the microscope apparatus 100, an average cell size may be calculated from the identified cell regions, and the size of the divided region A may be set on the basis of the calculated cell size.

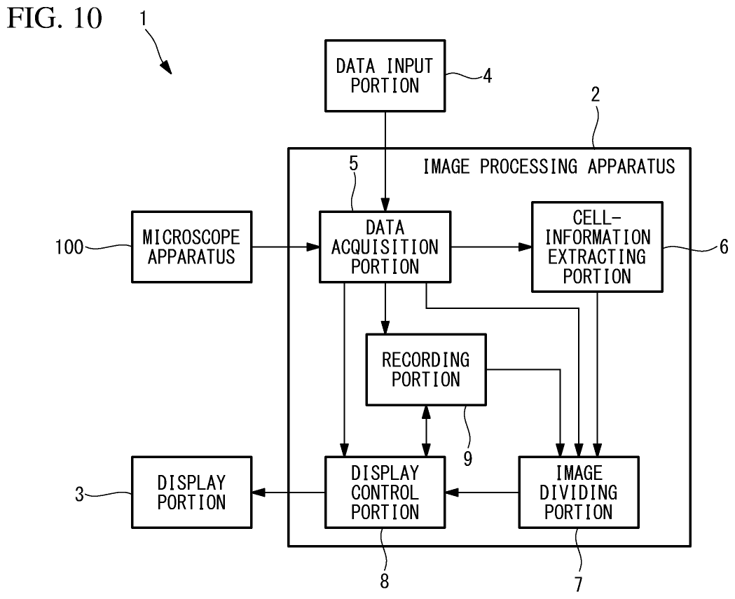

In addition, this embodiment may include, as shown in FIG. 10, a recording portion 9 that records a work history containing past images G that have been acquired by the data acquisition portion 5 and in which the annotations have been input or the images G in which annotation input is in progress.

The recording portion 9 records operator identification information, the metadata (the cell type, container, lot, the number of passages, the image magnification, the size of real viewing field, and so forth) accompanying the image G, the number by which the image G is divided, measured values of cell sizes, and so forth in association with the images before the division and the images after the division. In addition, as the work history in which the input is in progress, such information as information indicating the degree to which the annotation work has been completed with respect to specific images G and specific divided regions A therein may be recorded.

When the operator inputs the identification information from the data input portion 4, the work history in which the work is in progress and that is recorded in the recording portion 9 in association with the identification information may be read out and the annotation work may be performed in continuation.

In addition, in the case in which a new image G is acquired, a search may be performed to determine whether or not there is a past work history in which the cell type, the cell size, and so forth are similar to those of the image G, and, in the case in which there is such a work history, the image dividing portion 7 may divide the image G by using the size of the divided region A used in the similar past work history.

In addition, when the operator inputs the identification information from the data input portion 4, the image dividing portion 7 may divide the image G by using the size of the divided region A used in the past work history that is recorded in the recording portion 9 in association with the identification information.

In the case in which the annotation work to be continued is performed, the case in which the annotation work is performed on the new image G, or the like, as a result of utilizing a division parameter such as the size of the divided region A used in the annotation work performed on similar cells X in the past, there is an advantage in that it is possible to start the annotation work earlier.

In addition, in the case in which multiple types of cells X are present in a mixed state, the image G may be divided into small divided regions in accordance with small cells X, large divided regions in accordance with large cells X, or intermediate divided regions having an intermediate size. Alternatively, these size classes may be sequentially switched.

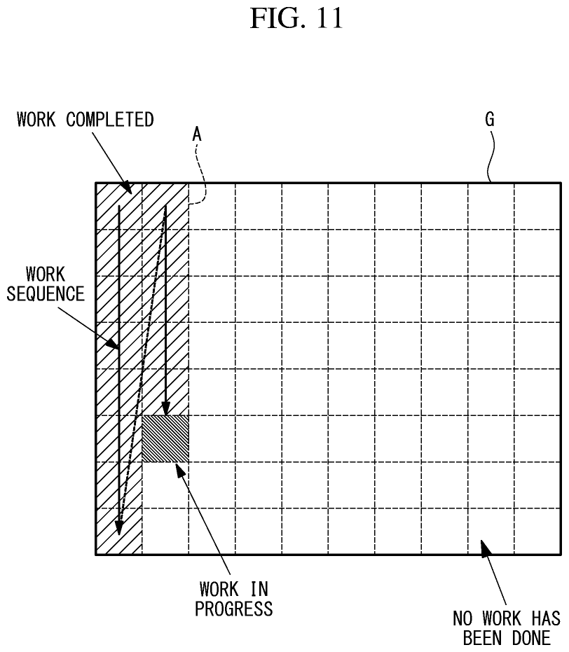

In addition, in order to execute the annotation work on all of the divided regions A formed by dividing the image G while reducing omissions in the annotation work, the display control portion 8 may sequentially display the divided regions A in a sequence set in advance. For example, the divided regions A may be displayed in the same sequence as indicated by arrows in FIG. 11 so that the work is sequentially performed from the upper left corner of the image G.

As a result of displaying the divided regions A in such a sequence, there is an advantage in that it is easy to ascertain the degree of progress of the annotation work in the overall image display.

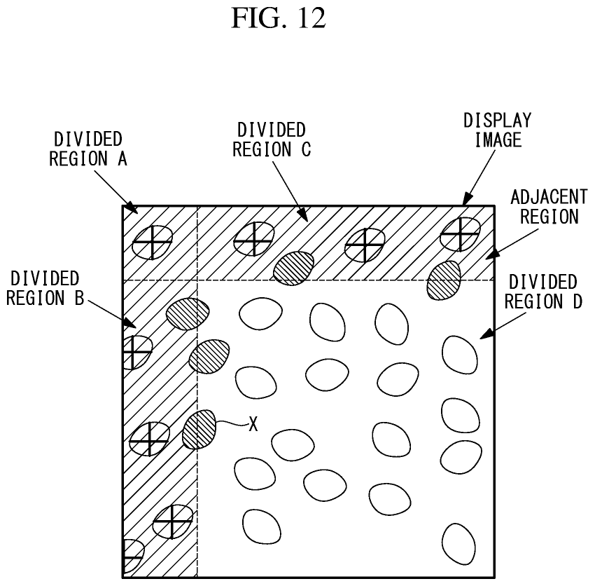

In addition, as a result of performing the annotation work by displaying the divided regions A in such a sequence from the upper left corner, a divided region D on which a new annotation work is to be performed is adjacent to the divided regions B and C, in which the work has been completed, on the left side and the top side of the divided region D, as shown in FIG. 12. Regarding the cells X that bridge across the boundary between the two divided regions B and C, there are cases in which the annotations are added and cases in which the annotations are not added; therefore, there are cases in which corrections are necessary when performing the annotation work in the new divided region D.

In this case, as a result of sequentially displaying the divided regions from the upper left corner, regions in which corrections are necessary are present only in the divided regions B on the left side and C on the top side; therefore, it is not necessary to review all directions, and there is an advantage in that it is possible to efficiently execute the annotation work while reducing omissions. In the case in which a divided region A is randomly selected, because the directions of the divided regions B and C that are adjacent to the new divided region D and in which the work has been completed are different for each divided region A, cases in which correction work is inadvertently omitted sometimes occur; however, it is possible to reduce the occurrence of such a problem by sequentially displaying the divided regions A.

In addition, in the case in which the plurality of cell types are present in a mixed state and the large divided regions and the small divided regions are present in a mixed state, it is possible to prevent inadvertent omission by generating the small divided regions by equally dividing the large divided regions and by sequentially displaying the small divided regions.

In addition, when the display control portion 8 displays the divided region D in which the annotation work is to be performed, as shown in FIG. 12, the divided region D may be displayed including adjacent regions that are portions of other divided regions B and C that are adjacent to the divided region D and in which the work has been completed. Accordingly, it is possible to prevent inadvertent omission in the annotation work on the cells X that are disposed bridging the adjacent divided regions B and C.

In the figure, the cells X indicated by hatching are the cells X disposed bridging the adjacent divided regions B, C, and D, and, regarding the annotation work in the divided regions B and C in which the work has been completed, the cells X remain without annotations. As a result of displaying the divided region D including the adjacent regions thereof, in the annotation work in the new divided region D, it is possible to perform the work to add the annotations to the cells X indicated by hatching or to correct the annotations thereof while reducing omissions.

In addition, in this embodiment, although the configuration in which the data-creation assistance method is realized by means of hardware has been described, the data-creation assistance method may be realized by means of a data-creation assistance program that can be executed by a computer. In this case, the computer includes a CPU, a principal recording apparatus such as a RAM, and a computer-readable recording medium, and the data-creation assistance program for realizing the above-described processing is stored in the recording medium. In addition, the CPU can realize the same processing as the data-creation assistance apparatus 1 by reading out the data-creation assistance program recorded in the recording medium.

The above-described embodiment also leads to the following aspects.

An aspect of the present invention is a data-creation assistance apparatus which is an apparatus that assists data creation work in which an annotation is added to each cell in an image containing cells, the data-creation assistance apparatus including: an image-data acquisition portion that acquires image data including the image; a cell-information extracting portion that extracts information related to a density of the cells from the acquired image data; an image dividing portion that sets a size of divided regions on the basis of the extracted information related to the density of the cells and that divides the image into the divided regions; and a display portion that displays each of the divided regions, which have been divided, on a single screen.

With this aspect, when the image-data acquisition portion acquires the image data, the cell-information extracting portion extracts the information related to the density of the cells from the image data, and the size of the divided regions is set on the basis of the extracted information related to the density of the cells. The cell density decreases with an increase in the cell size and an operator can visually recognize the cells even if the size of the divided regions is large. On the other hand, the cell density increases with a decrease in the cell size and it becomes difficult for the operator to visually recognize the cells unless the size of the divided regions is decreased and the display magnification is increased. Therefore, as a result of setting the size of the divided regions on the basis of the information related to the density of the cells, it is possible to display the cells in the size that facilitates visual recognition by the operator, and it is not necessary to frequently enlarge and shrink the image in the data creation work in which the annotation is added to each cell. Accordingly, it is possible to perform the annotation work at a constant quality by reducing omissions in the annotation work.

In the above-described aspect, the information related to the density of the cells may include the density of the cells and at least one of a size of the cells and a type of the cells.

With this configuration, the divided regions are displayed at a size that facilitates visual recognition by the operator on the basis of at least one of the cell size and the cell type in addition to the cell density, and thus, it is possible to perform the annotation work at a constant quality by reducing omissions in the annotation work.

In addition, the above-described aspect may include a recording portion that records a work history of the data creation work, wherein, in a case in which a current data creation work is similar to a past work history recorded in the recording portion, the image dividing portion may divide the image by using the size of the divided regions used in the similar past work history.

With this configuration, the image is divided by using the size of the divided regions used in the similar past work history.

In addition, the above-described aspect may include an input portion to which identification information of an operator who performs the data creation work is input, wherein the work history may include the identification information, and the image dividing portion may divide the image by using the size of the divided regions used in the past work history including the identification information.

With this configuration, the image is displayed in a divided manner by using the size of the divided regions used in the past work history of the same operator. In the case in which the size of the divided regions that facilitates the work is different for each operator, it is possible to enhance the workability by displaying the image in a divided manner at the size of the divided regions used in the past work of the same operator.

In addition, in the above-described aspect, the work history may include the information related to the density of the cells, and the image dividing portion may divide the image by using the size of the divided regions used in the past work history including the same information as the information related to the density of the cells extracted by the cell-information extracting portion.

With this configuration, when the cell-information extracting portion extracts the information related to the density of the cells, the past work history containing the same information is searched for in the past work history recorded in the recording portion, and the image is divided by using the size of the divided regions used in the past work history.

In addition, in the above-described aspect, the display portion may sequentially display the divided regions in accordance with a sequence set in advance.

With this configuration, in the annotation work applied to the image divided into the plurality of divided regions, it is easy to ascertain the degree of progress and it is possible to ascertain the additionally required work time.

In addition, in the above-described aspect, when displaying the divided regions, the display portion may display a region that includes at least adjacent regions of other divided regions in which the data creation work has already been performed and that are adjacent to the divided regions.

With this configuration, it is possible to prevent inadvertent omission in the annotation work on cells that are disposed bridging across the adjacent divided regions.

Another aspect of the present invention is a data-creation assistance method that assists data creation work in which an annotation is added to each cell in an image containing cells, the data-creation assistance method including: acquiring image data including the image; extracting information related to a density of the cells from the acquired image data; setting a size of divided regions on the basis of the extracted information related to the density of the cells; dividing the image into the divided regions; and displaying each of the divided regions, which have been divided, on a single screen.

Another aspect of the present invention is a data-creation assistance program that assists data creation work in which an annotation is added to each cell in an image containing cells, the data-creation assistance program causing a computer to execute: a step of acquiring image data including the image; a step of extracting information related to a density of the cells from the acquired image data; a step of setting a size of divided regions on the basis of the extracted information related to the density of the cells; a step of dividing the image into the divided regions; and a step of displaying each of the divided regions, which have been divided, on a single screen.

### REFERENCE SIGNS LIST

1 data-creation assistance apparatus
3 display portion
4 data input portion (input portion)
5 data acquisition portion (image-data acquisition portion)
6 cell-information extracting portion
7 image dividing portion
9 recording portion
A, B, C, D divided region
G image
X cell

1. A data-creation assistance apparatus which is an apparatus that assists data creation work in which an annotation is added to each cell in an image containing cells, the data-creation assistance apparatus comprising:
a processor comprising hardware, the processor being configured to:
acquire image data including the image;
extract information related to the cells from the acquired image data;
set a size of divided regions on the basis of the extracted information related to the cells; and
divide the image into the divided regions; and
a display that displays each of the divided regions which have been divided.

2. The data-creation assistance apparatus according to claim 1, wherein the information related to the cells includes at least one of a density of the cells, a size of the cells, and a type of the cells.

3. The data-creation assistance apparatus according to claim 1, wherein the processor is configured to extract the information related to the cells by applying image processing to the image.

4. The data-creation assistance apparatus according to claim 1, wherein the image data further includes metadata, and
the processor is configured to extract the information related to the cells by comparing the metadata with past data.

5. The data-creation assistance apparatus according to claim 1, further comprising a memory that records a work history of the data creation work,
wherein, in a case in which current data creation work is similar to a past work history recorded in the memory, the processor is configured to divide the image by using the size of the divided regions used in the similar past work history.

6. The data-creation assistance apparatus according to claim 5,
wherein the work history includes identification information of an operator who performs the data creation work, and
the processor is configured to divide the image by using the size of the divided regions used in the past work history including the identification information.

7. The data-creation assistance apparatus according to claim 5, wherein the work history includes the information related to the cells, and
the processor is configured to divide the image by using the size of the divided regions used in the past work history including the same information as the extracted information related to the cells.

8. The data-creation assistance apparatus according to claim 1, wherein the display sequentially displays the divided regions in accordance with a sequence set in advance.

9. The data-creation assistance apparatus according to claim 8, wherein, when displaying the divided regions, the display displays a region that includes at least adjacent regions of other divided regions in which the data creation work has already been performed and that are adjacent to the divided regions.

10. The data-creation assistance apparatus according to claim 1, wherein the display displays the added annotation on the divided regions in a superimposed manner.

11. The data-creation assistance apparatus according to claim 1, wherein the processor is configured to switches the size of the divided regions.

12. The data-creation assistance apparatus according to claim 1, wherein the display distinguishably displays the divided regions in which the data creation work is performed and the divided regions in which the data creation work is not performed.

13. A data-creation assistance method that assists data creation work in which an annotation is added to each cell in an image containing cells, the data-creation assistance method comprising:
acquiring image data including the image;
extracting information related to the cells from the acquired image data;
setting a size of divided regions on the basis of the extracted information related to the cells;
dividing the image into the divided regions; and
displaying each of the divided regions which have been divided.

14. A non-transitory computer-readable medium having a data-creation assistance program stored therein, the data-creation assistance program assisting data creation work in which an annotation is added to each cell in an image containing cells and causing a computer to execute function of:
acquiring image data including the image;
extracting information related to the cells from the acquired image data;
setting a size of divided regions on the basis of the extracted information related to the cells;
dividing the image into the divided regions; and
displaying each of the divided regions which have been divided.
